(19) United States
(12) Patent Application Publication
(10) Pub. No.: US 2013/0142815 A1
(43) Pub. Date: Jun. 6, 2013

(54) ALPHA-METHYL-TRYPTOPHAN AS AN INHIBITOR OF INDOLEAMINE DIOXYGENASE

(71) Applicant: Vadivel Ganapathy, Martinez, GA (US)

(72) Inventor: Vadivel Ganapathy, Martinez, GA (US)

(73) Assignee: Georiga Health Sciences Univ. Research Inst., Inc., Augusta, GA (US)

(21) Appl. No.: 13/577,927

(22) PCT Filed: Feb. 9, 2011
(86) PCT No.: PCT/US11/24155

§ 371 Date: Jan. 28, 2013

Related U.S. Application Data
(60) Provisional application No. 61/302,602, filed on Feb. 9, 2010.

(51) Int. Cl. A61K 31/405; A61K 39/39; A61N 5/00; A61K 45/06

(52) U.S. Cl. CPC A61K 31/405; A61K 45/06; A61K 39/39; A61N 5/00
USPC 424/184.1; 514/419; 435/184; 424/278.1; 600/1

(57) ABSTRACT

The present invention demonstrates for the first time that alpha-methyl-tryptophan is an inhibitor of the enzyme indoleamine diooxygenase (IDO). The present invention includes the use of alpha-methyl-tryptophan in methods of modulating immune responses and treating cancer and infections.

This application claims the benefit of U.S. Provisional Application Ser. No. 61/302,602, filed Feb. 9, 2010, which is incorporated by reference herein.

### BACKGROUND

The successful immunotherapy of cancer will require a combination of multiple immunomodulatory agents and strategies (Pure et al., 2005, Nat Immunol; 6:1207-1210). The immunoregulatory enzyme indoleamine 2,3-dioxygenase (IDO) is one molecular mechanism that contributes to tumor-induced tolerance. IDO helps create a tolerogenic milieu in the tumor and the tumor-draining lymph nodes, both by direct suppression of T cells and enhancement of local Treg-mediated immunosuppression. It can also function as an antagonist to other activators of antitumor immunity. Therefore, strategies to block IDO will enhance the effectiveness of tumor immunotherapy (Munn and Mellor, 2007, J Clin Invest; 117(5):1147-54)

### SUMMARY

The present invention includes a method of inhibiting indoleamine-2,3-dioxygenase (IDO), the method including contacting the IDO with a composition including alpha-methyl-tryptophan.

The present invention includes a method of inhibiting indoleamine-2,3-dioxygenase (IDO) in a subject, the method including administering an effective amount of a composition including alpha-methyl-tryptophan.

The present invention includes a method of treating cancer in a subject, the method including administering to the subject an effective amount of a composition including alpha-methyl-tryptophan. In some aspects, the cancer includes melanoma, colon cancer, pancreatic cancer, breast cancer, prostate cancer, lung cancer, leukemia, brain tumors, lymphoma, sarcoma, ovarian cancer, or Kaposi's sarcoma. In some aspects, the cancer does not express the ATB^{0,+} transporter. In some aspects, the cancer expresses the ATB^{0,+} transporter.

The present invention includes a method of increasing T cell activation by an antigen-bearing cell, the method including administering an effective amount of a composition including alpha-methyl-tryptophan.

The present invention includes a method of enhancing an immune response, the method including administering an effective amount of a composition including alpha-methyl-tryptophan.

The present invention includes a method to enhance an immune response to an antigen in a subject, the method including administering to the subject an effective amount of such an antigen in combination with alpha-methyl-tryptophan.

The present invention includes a method of reducing immune suppression mediated by regulatory T cells (Tregs) in a subject, the method including administering to the subject an effective amount of a composition including alpha-methyl-tryptophan.

The present invention includes a method of enhancing a T cell mediated immune response, the method including administering an effective amount of a composition including alpha-methyl-tryptophan.

The present invention includes a method of treating a subject with an infection, the method including administering to the subject an effective amount of a composition including alpha-methyl-tryptophan.

The present invention includes a method of treating a subject receiving a bone marrow transplant or peripheral blood stem cell transplant, the method including administering alpha-methyl-tryptophan.

The present invention includes a method of supplementing standard antiretroviral HIV therapy, the method including administering an effective amount of a composition including alpha-methyl-tryptophan to an individual already undergoing a standard antiretroviral HIV therapy.

The present invention includes a method of treating a subject with a cancer or an infection, the method including administering to the subject alpha-methyl-tryptophan in an amount effective to reverse indoleamine-2,3-dioxygenase-mediated immunosuppression, and administering at least one additional therapeutic agent.

In some aspects of the methods of the present invention, the subject is an individual with cancer. In some aspects, the cancer includes melanoma, colon cancer, pancreatic cancer, breast cancer, prostate cancer, lung cancer, leukemia, brain tumors, lymphoma, sarcoma, ovarian cancer, or Kaposi's sarcoma. In some aspects, the cancer does not express the ATB^{0,+} transporter. In some aspects, the cancer expresses the ATB^{0,+} transporter.

In some aspects of the methods of the present invention, the subject is an individual with HIV.

In some aspects of the methods of the present invention, alpha-methyl-tryptophan is the isolated D isomer, is the isolated L isomer, or includes a racemic mixture. In some aspects of the methods of the present invention, the alpha-methyl-tryptophan includes the D isomer of alpha-methyl-tryptophan and does not include the L isomer of alpha-methyl tryptophan. In some aspects, alpha-methyl-tryptophan is the L isomer of alpha-methyl-tryptophan and is not the D isomer of alpha-methyl tryptophan.

In some aspects of the methods of the present invention, alpha-methyl-tryptophan is formulated for controlled or sustained release. In some aspects, a formulation for controlled or sustained release is suitable for subcutaneous implantation. In some aspects, a formulation for controlled or sustained release includes a patch. In some aspects, alpha-methyl-tryptophan is formulated for enteral administration. In some aspects, alpha-methyl-tryptophan is formulated for systemic administration. In some aspects, alpha-methyl-tryptophan is formulated as a capsule or tablet. In some aspects, alpha-methyl-tryptophan is formulated for topical administration.

In some aspects of the methods of the present invention, a method further includes the administration of one or more additional therapeutic agents.

In some aspects of the methods of the present invention, an additional therapeutic agent includes an antiviral agent, an antibiotic, an antimicrobial agent, a cytokine, a vaccine, or combinations thereof.

In some aspects of the methods of the present invention, the administration of alpha-methyl-tryptophan and the at least one additional therapeutic agent demonstrate therapeutic synergy. In some aspects of the methods of the present invention, a measurement of response to treatment observed after administering both alpha-methyl-tryptophan and the additional therapeutic agent is improved over the same measurement of response to treatment observed after administering either the alpha-methyl-tryptophan or the additional therapeutic agent alone.

In some aspects of the methods of the present invention, at least one additional therapeutic agent includes an antineoplastic chemotherapy agent. In some aspects, an antineoplastic chemotherapeutic agent includes cyclophosphamide, methotrexate, fluorouracil, doxorubicin, vincristine, ifosfamide, cisplatin, gemcytabine, busulfan, ara-C, or combinations thereof.

In some aspects of the methods of the present invention, at least one additional therapeutic agent includes radiation therapy. In some aspects, radiation therapy includes localized radiation therapy delivered to the tumor. In some aspects, radiation therapy includes total body irradiation.

In some aspects of the methods of the present invention, an infection includes a viral infection, infection with an intracellular parasite, or infection with an intracellular bacteria. In some aspects, the viral infection includes human immunodeficiency virus or cytomegalovirus. In some aspects, the intracellular parasite includes Leishmania donovani, Leishmania tropica, Leishmania major, Leishmania aethiopica, Leishmania mexicana, Plasmodium falciparum, Plasmodium vivax, Plasmodium ovale, or Plasmodium malariae. In some aspects, the intracellular bacteria includes Mycobacterium leprae, Mycobacterium tuberculosis, Listeria monocytogenes, or Toxplasma gondii.

In some aspects of the methods of the present invention, at least one additional therapeutic agent includes an inhibitor of indoleamine-2,3-dioxygenase (IDO) other than alpha-methyl-tryptophan.

In some aspects of the methods of the present invention, at least one additional therapeutic agent includes an inhibitor of the ATB^{0,+} amino acid transporter other than alpha-methyl-tryptophan.

In some aspects of the methods of the present invention, the at least one additional therapeutic agent includes 1-methyl-tryptophan (1-MT). In some aspects, the 1-MT is the isolated D isomer of 1-MT, is the isolated L isomer of 1-MT, or includes a racemic mixture of 1-MT.

In some aspects of the methods of the present invention, the at least one additional therapeutic agent includes a TLR9 agonist. In some aspects, the TLR9 agonist includes a CpG-oligonucleotide.

In some aspects of the methods of the present invention, the at least one additional therapeutic agent is one or more inhibitors of the CTLA4 pathway. In some aspects, an inhibitor of the CTLA4 pathway includes one or more antibodies against CTLA4.

In some aspects of the methods of the present invention, the at least one additional therapeutic agent includes one or more inhibitors of the PD-1/PD-L pathway. In some aspects, the one or more inhibitors of the PD-1/PD-L pathway include one or more antibodies against PD-1, PD-L1, and/or PD-L2.

The present invention includes a composition for inhibiting indoleamine-2,3-dioxygenase (IDO), the composition including alpha-methyl-tryptophan. The present invention includes a composition for treating a subject with cancer, the composition including alpha methyl-tryptophan. The present invention includes a composition for treating a subject with an infection, the composition including alpha methyl-tryptophan. The present invention includes a composition to enhance an immune response, the composition including alpha methyl-tryptophan. The present invention includes a composition to reduce immune suppression mediated by regulatory T cells (Tregs) in a subject, the composition including alpha-methyl-tryptophan. In some aspects of a composition, alpha-methyl-tryptophan is the isolated D isomer, is the isolated L isomer, or includes a racemic mixture. In some aspects, a composition includes the D isomer of alpha-methyl-tryptophan and does not include the L isomer of alpha-methyl-tryptophan. In some aspects, a composition includes the L isomer of alpha-methyl-tryptophan and does not include the D isomer of alpha-methyl-tryptophan. In some aspects, a composition further includes at least one additional therapeutic agent. In some aspects, the at least one additional therapeutic agent includes an inhibitor of indoleamine-2,3-dioxygenase other than alpha-methyl-tryptophan. In some aspects, the at least one additional therapeutic agent includes 1-methyl-tryptophan (1-MT). In some aspects, the 1-MT is the isolated D isomer of 1-MT, is the isolated L isomer of 1-MT, or includes a racemic mixture of 1-MT. In some aspects, the at least one additional therapeutic agent includes an antigen, a TLR9 agonist, a vaccine, a cytokine, one or more inhibitors of the PD-1/PD-L pathway, and/or one or more inhibitors of the CTLA4 pathway. In some aspects, a composition is formulated for controlled or sustained release. In some aspects, a composition formulated for controlled or sustained release is suitable for subcutaneous implantation. In some aspects, a composition formulated for controlled or sustained release is a patch. In some aspects, a composition is formulated for enteral administration. In some aspects, a composition is formulated for systemic administration. In some aspects, a composition is formulated as a capsule or tablet. In some aspects, a composition is formulated for topical administration.

The term “and/or” means one or all of the listed elements or a combination of any two or more of the listed elements.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.).

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

For any method disclosed herein that includes discrete steps, the steps may be conducted in any feasible order. And, as appropriate, any combination of two or more steps may be conducted simultaneously.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1 presents the comparative potencies of D-, L-, and DL-isomers of 1-methyltryptophan (1-MT) and alpha-methyltr tophan (alpha-MT) for inhibition of indoleamine dioxygenase (IDO).

FIG. 2 presents the optical resolution method used to separate the L- and D-isomers of α-methyltryptophan from a commercially available DL-racemic mixture of α-methyltryptophan.

### DETAILED DESCRIPTION OF ILLUSTRATIVE EMBODIMENTS

The present disclosure demonstrates for the first time that alpha-methyl-tryptophan (also referred to herein as alpha-methyltryptophan, alpha-MT, alpha MTrp, α-methyltryptophan, α-methyl-tryptophan, a-MT, aMT, aMTrp, α-methyltryptophan, α-methyl-tryptophan, α-MT, αMT, or α-MTrp) is an inhibitor of the enzyme indoleamine diooxygenase (IDO). These findings indicate that alpha-methyl-tryptophan will be effective in vivo as an immunomodulating agent and as an anti-cancer agent. The present invention includes compositions of alpha-methyltryptophan and the use of alpha-methyltryptophan in methods of modulating immune responses and treating cancer and infections.

A recently recognized molecular mechanism contributing to peripheral immune tolerance is the immunoregulatory enzyme indoleamine 2,3-dioxygenase (IDO). Cells expressing the tryptophan-catabolizing enzyme IDO are capable of inhibiting T cell proliferation in vitro and reducing T cell immune responses in vivo (see, for example, U.S. Pat. Nos. 6,451,840 and 6,482,416; Munn et al., 1998, Science; 281:1191; Munn et al. 1999, J Exp Med; 189:1363; Hwu et al., 2000, J Immunol; 164:3596; Mellor et al., 2002, J Immunol; 168:3771; Grohmann et al., 2001, J Immunol; 167:708; Grohmann et al., 2001, J Immunol; 166:277; and Alexander et al., 2002, Diabetes; 51:356). IDO degrades the essential amino acid tryptophan (for reviews see Taylor et al., 1991, FASEB Journal; 5:2516-2522; Lee et al., 2003, Laboratory Investigation: 1457-1466; and Grohmann et al., 2003, Trends in Immunology; 24:242-248). Expression of IDO by human monocyte-derived macrophages (Munn et al., 1999 J Exp Med; 189:1363-1372), human dendritic cells (Munn et al., 2002, Science; 297:1867-1870 and Hwu et al., 2000, J Immunol; 164:3596-3599), and mouse dendritic cells (Mellor et al., 2003, J Immunol; 171:1652-1655) allows these different antigen-presenting cells (APCs) to inhibit T cell proliferation in vitro.

IDO has also been implicated in maintaining tolerance to self antigens (Grohmann et al., 2003, J Exp Med; 198:153-160), in suppressing T cell responses to MHC-mismatched organ transplants (Miki et al., 2001, Transplantation Proceedings; 33:129-130; Swanson, et al. 2004, Am J Respir Cell Mol Biol; 30:311-8; Beutelspacher et al., 2006, Am J Transplant; 6:1320-30) and in the tolerance-inducing activity of recombinant CTLA4-Ig (Grohmann et al. 2002, Nature Immunology; 3:985-1109; Mellor et al., 2003, J Immunol; 171:1652-1655) and the T cell regulatory functions of interferons (Grohmann et al., 2001, J Immunol; 167:708-14; and Baban et al., 2005, Int Immunol; 17:909-919). In these systems, the immunosuppressive effect of IDO can be blocked by the in vivo administration of the IDO inhibitor 1-methyl-tryptophan (also referred to herein as 1-MT or 1MT) and the IDO inhibitor 1-methyl-tryptophan is being developed for clinical trials (see, for example, Hou et al., 2007, Cancer Res; 67(2):792-801).

The IDO enzyme is well characterized (see, for example, Taylor et al., 1991, FASEB J; 5:2516-2522; Lee et al., 2003, Laboratory Investigation; 83:1457-1466; and Grohmann et al., 2003, Trends Immunol; 24:242-248) and compounds that serve as substrates or inhibitors of the IDO enzyme are known. Southan (Southan et al., 1996, Med. Chem. Res; 343-352) utilized an in vitro assay system to identify tryptophan analogues that serve as either substrates or inhibitors of human IDO. Methods for detecting the expression and function of IDO are well known and include, but are not limited to, any of those described herein and those described, for example in U.S. Pat. Nos. 6,395,876, 6,451,840, and 6,482,416, U.S. Patent Application Nos. 20030194803, 20040234623, 20050186289, and 20060292618, PCT/US2006/040796, and PCT/US2007/000404.

The present invention includes the use of alpha-methyl-tryptophan to inhibit indoleamine-2,3-dioxygenase (IDO). With the present invention, alpha-methyl-tryptophan may be administered to a subject in an amount effective to inhibit or reduce the function of the IDO. With the present invention, alpha-methyl-tryptophan may be administered to a subject in an amount effective to reverse indoleamine-2,3-dioxygenase-mediated immunosuppression. With the present invention, alpha-methyl-tryptophan may be administered to a subject in an amount effective to treat cancer or an infection. Alpha-methyl-tryptophan may used, for example, in methods of treating cancer and infections in a subject, methods of treating a subject receiving a bone marrow transplant or peripheral blood stem cell transplant, and methods of supplementing standard antiretroviral HIV therapy.

With the present invention, alpha-methyl-tryptophan may be used in any of a variety of methods of immunomodulation, including, but not limited to, methods of increasing T cell activation by an antigen-bearing cell, methods of enhancing an immune response, methods of reducing immune suppression mediated by regulatory T cells (Tregs), methods of enhancing a T cell mediated immune response.

In addition to the demonstration of the present invention, that α-methyltryptophan is an inhibitor of (IDO), it has recently been shown that α-methyltryptophan functions as an inhibitor of the amino acid transporter ATB^{0,+} (see, for example, WO 2010/042685 and Karunakaran et al., 2008, Biochem J; 414:343-355). The amino acid transporter ATB^{0,+} (also referred to herein as “ATB^{0,+} transporter,” “the ATB0,+ transporter,” “ATB0,+,” “'ATB0,+,” “ATB(0,+),” “ATB (0,+),” and “SLC6A14”) is an amino acid transporter with special functional features (Ganaphthy and Ganaphthy, 2005, Curr. Drug Targets Immune Endocr Metab Disord; 5:357-364). There is emerging evidence for tumor-associated up-regulation of ATB^{0,+} (Gupta et al., 2005, Biochim Biophys Acta; 1741:215-223; Gupta et al., 2006, Gynecol Oncol; 100:8-13). The expression of this transporter is markedly induced in colorectal cancer (Gupta et al., 2005, Biochim Biophys Acta; 1741:215-223) and cervical cancer (Gupta et al., 2006, Gynecol Oncol; 100:8-13), and the up-regulation is demonstrable at the mRNA level as well as at the protein level.

In some embodiments of the present invention, the administration of alpha-methyl-tryptophan may have the additional effect of inhibiting the ATB^{0,+} transporter. When administered to a subject with a cancer that expresses the ATB^{0,+} transporter, the administration of alpha-methyl-tryptophan may demonstrate a synergistic effect, targeting two molecules; the ATB^{0,+} transporter in tumor cells and IDO in tumor-associated immune cells. Alpha-methyl-tryptophan may produce anti-cancer effects in vivo by two different, but synergistic, mechanisms. One, blocking the transport functions of the ATB^{0,+} transporter in tumor cells, thus preventing entry of essential amino acids and glutamine, and consequently causing growth arrest and apoptosis. And, two, inhibiting IDO in tumor-associated immune cells, thus disrupting tolerance toward tumors and consequently triggering anti-tumor immune responses.

In some embodiments of the present invention, a racemic mixture of alpha-methyl-tryptophan may be used. In some embodiments, an isolated D isomer of alpha-methyl-tryptophan may be used, that is a composition that includes the D isomer but does not include the L isomer may be used. In some embodiments, an isolated L isomer of alpha-methyl-tryptophan may be used, that is, a composition that includes the L isomer but does not include the D isomer may be used. For example, a mixture of the D and L isomers, including, for example, a racemic mixture, may be used. A composition that includes one enantiomer, but does not include the other enantiomer, may be used. For example, a composition that includes the D isomer, but does not include the L isomer, may be used. Such a composition consists essentially of the D isomer of alpha-methyl-tryptophan. A composition that includes the L isomer, but does not include the D isomer, may be used. Such a composition consists essentially of the L isomer of alpha-methyl-tryptophan. The purification of D and L isomers can be carried out, for example, as described herein in Example 3

One or more additional therapeutic modalities may be administered along with the present methods of administering alpha-methyl-tryptophan. In some aspects of the present invention, the administration of alpha-methyl-tryptophan may allow for the effectiveness of a lower dosage of other therapeutic modalities when compared to the administration of the other therapeutic modalities alone, providing relief from the toxicity observed with the administration of higher doses of the other modalities. One or more additional therapeutic agents may be administered before, after, and/or coincident to the administration of alpha-methyl-tryptophan. Alpha-methyl-tryptophan and additional therapeutic agents may be administered separately or as part of a mixture of cocktail. As used herein, an additional therapeutic agent is an agent whose use for the treatment of cancer, an infection, or immune modulation is known to the skilled artisan. As used herein, an additional therapeutic agent is not alpha-methyl-tryptophan. Additional therapeutic treatments include, but are not limited to, surgical resection, radiation therapy, hormone therapy, vaccines, antibody based therapies, whole body irradiation, bone marrow transplantation, peripheral blood stem cell transplantation, the administration of chemotherapeutic agents (also referred to herein as “antineoplastic chemotherapy agent,” “antineoplastic agents,” or “antineoplastic chemotherapeutic agents”), cytokines, antiviral agents, immune enhancers, tyrosine kinase inhibitors, signal transduction inhibitors, antibiotic, antimicrobial agents, a TLR agonists, such as for example, bacterial lipopolysaccharides (LPS), one or more CpG oligonucleotides (ODN), metabolic breakdown products of tryptophan, inhibitors of a GCN2 kinase, inhibitors of the PD-1/PD-L pathway, inhibitors of the CTLA4 pathway, an inhibitor of IDO other than alpha-methyl-tryptophan, such as, for example, 1-MT, and adjuvants.

A chemotherapeutic agent may be, for example, a cytotoxic chemotherapy agent, such as, for example, epidophyllotoxin, procarbazine, mitoxantrone, platinum coordination complexes such as cisplatin and carboplatin, leucovorin, tegafur, paclitaxel, docetaxol, vincristine, vinblastine, methotrexate, cyclophosphamide, gemcitabine, estramustine, carmustine, adriamycin (doxorubicin), etoposide, arsenic trioxide, irinotecan, epothilone derivatives, navelbene, CPT-11, anastrazole, tetrazole, capecitabine, reloxafine, ifosamide, and droloxafine.

A chemotherapeutic agent may be, for example, an alkylating agent, such as, for example, nitrogen mustards (such as chlorambucil, cyclophosphamide, ifosfamide, echlorethamine, melphalan, and uracil mustard), aziridines (such as thiotepa), methanesulphonate esters (such as busulfan), nitroso ureas (such as carmustine, lomustine, and streptozocin), platinum complexes (such as cisplatin and carboplatin), and bioreductive alkylators (such as mitomycin, procarbazine, dacarbazine and altretamine), ethylenimine derivatives, alkyl sulfonates, triazenes, pipobroman, temozolomide, triethylene-melamine, and triethylenethiophosphoramine.

A chemotherapeutic agent may be an antimetabolite, such as, for example, a folate antagonist (such as methotrexate and trimetrexate), a pyrimidine antagonist (such as fluorouracil, fluorodeoxyuridine, CB3717, azacitidine, cytarabine, gemcitabine, and floxuridine), a purine antagonist (such as mercaptopurine, 6-thioguanine, fludarabine, and pentostatin), a ribonucleotide reductase inhibitor (such as hydroxyurea), and an adenosine deaminase inhibitor.

A chemotherapeutic agent may be a DNA strand-breakage agent (such as, for example, bleomycin), a topoisomerase II inhibitor (such as, for exmaple, amsacrine, dactinomycin, daunorubicin, idarubicin, mitoxantrone, doxorubicin, etoposide, and teniposide), a DNA minor groove binding agent (such as, for example, plicamydin), a tubulin interactive agent (such as, for example, vincristine, vinblastine, and paclitaxel), a hormonal agent (such as, for example, estrogens, conjugated estrogens, ethinyl estradiol, diethylstilbesterol, chlortrianisen, idenestrol, progestins (such as hydroxyprogesterone caproate, medroxyprogesterone, and megestrol), and androgens (such as testosterone, testosterone propionate, fluoxymesterone, and methyltestosterone)), an adrenal corticosteroid (such as, for example, prednisone, dexamethasone, methylprednisolone, and prednisolone), a leutinizing hormone releasing agent or gonadotropin-releasing hormone antagonist (such as, for example, leuprolide acetate and goserelin acetate), an antihormonal agent (such as, for example, tamoxifen), an antiandrogen agent (such as flutamide), an antiadrenal agent (such as mitotane and aminoglutethimide), and a natural product or derivative thereof (such as, for example, vinca alkaloids, antibiotics, enzymaes and epipodophyllotoxins, including, for example vinblastine, vincristine, vindesine, bleomycin, dactinomycin, daunorubicin, doxorubicin, epirubicin, idarubicin, ara-C, paclitaxel, mithramycin, deoxyco-foHnycin, mitomycin-C, L-asparaginase, and teniposide.

Antiviral agents include, but are not limited to, acyclovir, gangcyclovir, foscarnet, ribavirin, and antiretrovirals. Antiretrovirals include, for example, nucleoside analogue reverse transcriptase inhibitors (such as, for example, azidothymidine (AZT), didanosine (ddI), zalcitabine (ddC), stavudine (d4T), lamivudine (3TC), abacavir (1592U89), adefovir dipivoxil (bis(POM)-PMEA), lobucavir (BMS-180194), BCH-10652, emitricitabine ((−)-FTC), beta-L-FD4, DAPD, ((−)-beta-D-2,6-diamino-purine dioxolane), and lodenosine (FddA)), non-nucleoside reverse transcriptase inhibitors (suc as, for example, nevirapine, delaviradine, efavirenz, PNU-142721, AG-1549, MKC-442, and (+)-calanolide A (NSC-675451)), nucleotide analogue reverse transcriptase inhibitors, protease inhibitors (such as, for example, saquinavir, ritonavir, indinavir, nelfnavir, amprenavir, lasinavir, DMP-450, BMS-2322623, ABT-378, and AG-1549) and other antivirals (such as, for example, hydroxyurea, ribavirin, IL-2, IL-12, and pentafuside).

Cytokines include, but are not limited to, IL-1α, IL-1β, IL-2, IL-3, IL-4, IL-6, IL-8, IL-9, IL-10, IL-12, IL-13, IL-15, IL-18, IL-19, IL-20, IFN-α, IFN-β, IFN-γ, tumor necrosis factor (TNF), transforming growth factor-β (TGF-β), granulocyte colony stimulating factor (G-CSF), macrophage colony stimulating factor (M-CSF), granulocyte-macrophage colony stimulating factor (GM-CSF), and or Flt-3 ligand. Vaccines include, but are not limited to, vaccines against various infectious diseases, anti-tumor vaccines and anti-viral vaccines. Antitumor vaccines include, but are not limited to, peptide vaccines, whole cell vaccines, genetically modified whole cell vaccines, recombinant protein vaccines or vaccines based on expression of tumor associated antigens by recombinant viral vectors. Antibody therapeutics, include, for example, trastuzumab (Herceptin) and antibodies to cytokines, such as IL-10 and TGF-β.

Signal transduction inhibitors (STI) include, for example, bcr/abl kinase inhibitors such as, for example, STI 571 (Gleevec), epidermal growth factor (EGF) receptor inhibitors such as, for example, kinase inhibitors (Iressa, SSI-774) and the antibody C225, her-2/neu receptor inhibitors such as, for example, trastuzumab and farnesyl transferase inhibitors (FTI) such as, for example, L-744,832, inhibitors of Akt family kinases or the Akt pathway, such as, for example, rapamycin, cell cycle kinase inhibitors such as, for example, flavopiridol and UCN-01, and phosphatidyl inositol kinase inhibitors such as, for example, LY294002.

GCN2 inhibitors, include, for example, a GCN2 blocking peptide, an antibody to GCN2 (both commercially available, for example, from Bethyl, Inc., Montgomery, Tex.) and small molecule inhibitors (including, for example, those discussed by Muller and Scherle, 2006, Nature Reviews Cancer; 6:613).

Inhibitors of the PD-1/PD-L pathway include, but are not limited to, antibodies, peptides, nucleic acid molecules (including, for example, an antisense molecule, a PNA, or an RNAi), peptidomimetics, small molecules, a soluble PD-1 ligand polypeptide, or a chimeric polypeptide (for example, a chimeric PD-1 ligand/Immunoglobulin molecule). An antibody may be an intact antibody, an antibody binding fragment, or a chimeric antibody. A chimeric antibody may include both human and non-human portions. An antibody may be a polyclonal or a moncoclonal antibody. An antibody may be a derived from a wide variety of species, including, but not limited to mouse and human. An antibody may be a humanized antibody. An antibody may be linked to another functional molecule, for example, another peptide or protein, a toxin, a radioisotype, a cytotoxic agent, cytostatic agent, a polymer, such as, for example, polyethylene glycol, polypropylene glycol or polyoxyalkenes. Any of a variety of PD-1, PD-L1, and/or PD-L2 antibodies may be used, including, but not limited to, any of those described herein and, for example, those commercially available from, for example, R&D Systems, Invitrogen, BioLegend, eBiosciences, or Acris Antibodies, and those described, for example, in U.S. Patent Application Serial Nos. 2002 0164600; 2004 0213795; 2004 0241745; 2006 0210567; 2007 0092504; 2007 0065427; and 2008 0025979 and U.S. Pat. No. 7,101,550. In some embodiments, humanized anti-PD-1, anti-PD-L1, and/or anti-PD-L2, anti-PD1 antibodies may be used.

Inhibitors of the CTLA4 pathway include, but are not limited to antibodies, peptides, nucleic acid molecules (including, for example, an antisense molecule, a PNA, or an RNAi), peptidomimetics, small molecules, a soluble CTLA4 ligand polypeptide, or a chimeric polypeptide (for example, a chimeric CTLA4 ligand/immunoglobulin molecule). An antibody may be an intact antibody, an antibody binding fragment, or a chimeric antibody. A chimeric antibody may include both human and non-human portions. An antibody may be a polyclonal or a monoclonal antibody. An antibody may be a derived from a wide variety of species, including, but not limited to mouse and human. An antibody may be a humanized antibody. An antibody may be linked to another functional molecule, for example, another peptide or protein, a toxin, a radioisotype, a cytotoxic agent, cytostatic agent, a polymer, such as, for example, polyethylene glycol, polypropylene glycol or polyoxyalkenes. In some embodiments, a mixture or cocktail of various inhibitors of the CTLA4 pathway may be administered. Any of a variety of antibodies may be used, including, but not limited to, any of those described herein and those commercially available from, for example, Medarex, Princeton, N.J. (Medarex MDX010); eBioscience, San Diego Calif. (clone 9H10) Abnova Corporation, Taipei City, Taiwan (CTLA4 monoclonal antibody (M08), clone 1F4 Catalog#: H00001493-M08 and CTLA4 polyclonal antibody (A01) Catalog#: H00001493-A01); RDI Division of Fitzgerald Industries Intl., Concord MA (mouse anti-human CTLA-4 antibodies clones BNI3.1 and ANC152.2 (J Immunol 151:3469; J Immunol; 155:1776; and J Immunol; 156:1047)); and BD Pharmingen (hamster anti-mouse CTLA4 IgG1; clone UC10-4F10-11; hybridoma HB-304T from ATCC). Anti-CTLA4 antibodies include, but are not limited to, those taught in U.S. Pat. Nos. 7,311,910; 7,307,064; 7,132,281; 7,109,003; 7,034,121; 6,984,720; and 6,682,736. In some embodiments, one or more anti-CTLA4 antibodies may be humanized.

IDO inhibitors other than alpha-methyl-tryptophan include, but are not limited to antibodies, peptides, nucleic acid molecules (including, for example, an antisense molecule, a PNA, or an RNAi), peptidomimetics, and small molecules. Small molecule inhibitors of IDO include, but are not limited to, any of a variety of commercially available IDO inhibitors, such as, but not limited to, 1-methyl-DL-tryptophan (Sigma-Aldrich; St. Louis, Mo.), β-(3-benzofuranyl)-DL-alanine (Sigma-Aldrich), beta-(3-benzo(b)thienyl)-DL-alanine (Sigma-Aldrich), 6-nitro-L-tryptophan (Sigma-Aldrich), indole 3-carbinol (LKT Laboratories; St. Paul, Minn.), 3,3′-diindolylmethane (LKT Laboratories), epigallocatechin gallate (LKT Laboratories), 5-Br-4-Cl-indoxyl 1,3-diacetate (Sigma-Aldrich), 9-vinylcarbazole (Sigma-Aldrich), acemetacin (Sigma-Aldrich), 5-bromo-DL-tryptophan (Sigma-Aldrich), and 5-bromoindoxyl diacetate (Sigma-Aldrich). Small molecule inhibitors of IDO include, for example, any of the competitive and noncompetitive inhibitors of DO discussed in Muller et al. (Muller et al. 2005, Expert Opin Thr Targets; 9:831-849). IDO inhibitors may include, but are not limited to, any of a variety of the small molecule inhibitors of IDO described in US Patent Applications Nos. 20060258719, 20070203140 (including, but not limited to various N-hydroxyguanidines compounds), 20070185165 (including, but not limited to, various N-hydroxyamidinoheterocycles compounds), 20070173524 (including, but not limited to, various brassilexin and brassinin derivatives), and 20070105907 (including, but not limited to, various brassilexin and brassinin derivatives), WO 2004/094409, PCT/US2004/005154, WO/2006/005185 (naphtoquinones derivatives), PCT/CA2005/001087, Gaspari et al., 2006, J Med Chem; 49:684-92 (brassinin derivatives), Muller et al., 2005, Nat. Med; 11:312-319, Peterson et al., 1993, Med Chem Res; 3:473-482 (substituted beta-carbolines), Sono et al., 1989, Biochemistry; 28:5392-9, Sono et al., 1996, Chem Rev; 96:2841, and Vottero et al., 2006, Biotechnol J; 1:282-288. IDO inhibitors include, for example, any of compounds taught in PCT/US2007/000404, including, but not limited to, compounds A-YY, and analogs and derivatives thereof. In some embodiments of the present invention, an IDO inhibitor may be a racemic mixture of an inhibitor, an isolated D isomer of an inhibitor, or an isolated L isomer of an inhibitor, including, but not limited to, a racemic mixture of 1-MT, an isolated D isomer of 1-MT, or an isolated L isomer of 1-MT.

In some embodiments of the methods of the present invention, the additional therapeutic agent is 1-methyl-tryptophan (1-MT). In some embodiments, 1-MT may be a racemic mixture of 1-MT, an isolated D isomer of 1-MT, or an isolated L isomer of 1-MT.

As used herein “treating” or “treatment” can include therapeutic and/or prophylactic treatments. Desirable effects of treatment include preventing occurrence or recurrence of disease, alleviation of symptoms, diminishment of any direct or indirect pathological consequences of the disease, decreasing the rate of disease progression, amelioration or palliation of the disease state, and remission or improved prognosis. The findings of the present disclosure can be used in methods that include, but are not limited to, methods for treating cancer, methods for treating an infection, methods for increasing an immune response, methods for reducing immunosuppression mediated by regulatory T cells, and methods for increasing and/or stimulating T cell mediated immune responses.

The agents of the present disclosure can be administered by any suitable means including, but not limited to, for example, oral, rectal, nasal, topical (including transdermal, aerosol, buccal and sublingual), vaginal, parenteral (including subcutaneous, intramuscular, intravenous and intradermal), intravesical, or injection into or around the tumor.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intraperitoneal, and intratumoral administration. In this connection, sterile aqueous media that can be employed will be known to those of skill in the art. Some variation in dosage will necessarily occur depending on the condition of the subject being treated. The person responsible for administration will, in any event, determine the appropriate dose for the individual subject. Moreover, for human administration, preparations should meet sterility, pyrogenicity, and general safety and purity standards as required by the FDA. Such preparation may be pyrogen-free.

For enteral administration, the inhibitor may be administered in a tablet or capsule, which may be enteric coated, or in a formulation for controlled or sustained release. Many suitable formulations are known, including polymeric or protein microparticles encapsulating drug to be released, ointments, gels, or solutions which can be used topically or locally to administer drug, and even patches, which provide controlled release over a prolonged period of time. These can also take the fowl of implants. Such an implant may be implanted within the tumor.

Therapeutically effective concentrations and amounts may be determined for each application herein empirically by testing the compounds in known in vitro and in vivo systems, such as those described herein, dosages for humans or other animals may then be extrapolated therefrom.

It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions and methods.

An agent of the present disclosure may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions and methods.

In some therapeutic embodiments, an “effective amount” of an agent is an amount that results in a reduction of at least one pathological parameter. Thus, for example, in some aspects of the present disclosure, an effective amount is an amount that is effective to achieve a reduction of at least about 10%, at least about 15%, at least about 20%, or at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%, compared to the expected reduction in the parameter in an individual not treated with the agent.

With the present disclosure, the stimulation or inhibition of an immune response may be measured by any of many standard methods well known in the immunological arts. As used herein, a mixed leukocyte response (MLR) is a well-known immunological procedure, for example, as described in the examples herein. As used herein, T cell activation by an antigen-presenting cell is measured by standard methods well known in the immunological arts. As used herein, a reversal or decrease in the immunosuppressed state in a subject is as determined by established clinical standards. As used herein, the improved treatment of an infection is as determined by established clinical standards. The determination of immunomodulation includes, but is not limited to, any of the various methods as described in the examples herein.

With the methods of the present disclosure, the efficacy of the administration of one or more agents may be assessed by any of a variety of parameters known in the art. This includes, for example, determinations of an increase in the delayed type hypersensitivity reaction to tumor antigen, determinations of a delay in the time to relapse of the post-treatment malignancy, determinations of an increase in relapse-free survival time, determinations of an increase in post-treatment survival, determination of tumor size, determination of the number of reactive T cells that are activated upon exposure to the vaccinating antigens by a number of methods including ELISPOT, FACS analysis, cytokine release, or T cell proliferation assays.

As used herein, the term “subject” includes, but is not limited to, humans and non-human vertebrates. In preferred embodiments, a subject is a mammal, particularly a human. A subject may be an individual. A subject may be an “individual,” “patient,” or “host. Non-human vertebrates include livestock animals, companion animals, and laboratory animals. Non-human subjects also include non-human primates as well as rodents, such as, but not limited to, a rat or a mouse. Non-human subjects also include, without limitation, chickens, horses, cows, pigs, goats, dogs, cats, guinea pigs, hamsters, mink, and rabbits.

As used herein “in vitro” is in cell culture and “in vivo” is within the body of a subject.

As used herein, the term “pharmaceutically acceptable carrier” refers to one or more compatible solid or liquid filler, diluents or encapsulating substances which are suitable for administration to a human or other vertebrate animal.

As used herein, “isolated” refers to material that has been either removed from its natural environment (e.g., the natural environment if it is naturally occurring), produced using recombinant techniques, or chemically or enzymatically synthesized, and thus is altered “by the hand of man” from its natural state.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

In some embodiments of the present invention, a derivative of alpha-methyl-tryptophan may be used. Examples of derivations include, without limitation, substitution of the methyl group on the α-carbon with an ethyl, propyl, butyl, pentyl, or longer carbon chain. The carbon chain may be straight or it may be branched. Optionally, the methyl group or the substituted carbon chain may be present on the β-carbon. Another example of a derivation of the inhibitor may include, without limitation, the addition of groups on the aromatic carbons. For example, a methyl or a longer carbon chain may be present on one or more of the C1, C2, C3, C4, C5, C6, or C7 carbon(s) of the indole ring. Alternatively, a hydroxyl (—OH) or a halogen (such as bromine, fluorine, chlorine, or iodine) may be present on one or more of the C1, C2, C3, C4, C5, C6, or C7 carbon(s) of the indole ring.

The present invention includes compositions of alpha-methyl-tryptophan. A composition may also include, for example, buffering agents to help to maintain the pH in an acceptable range or preservatives to retard microbial growth. Such compositions may also include a pharmaceutically acceptable carrier. As used herein, the term “pharmaceutically acceptable carrier” refers to one or more compatible solid or liquid filler, diluents or encapsulating substances which are suitable for administration to a human or other vertebrate animal. The compositions of the present disclosure are formulated in pharmaceutical preparations in a variety of forms adapted to the chosen route of administration

The present disclosure also includes pharmaceutically acceptable salts of inhibitors. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. The pharmaceutically acceptable salts can be synthesized from the parent compound which contains a basic or acidic moiety by conventional chemical methods.

The methods of the present disclosure may be administered to a patient for the treatment of cancer. Cancers to be treated include, but are not limited to, melanoma, basal cell carcinoma, colorectal cancer, pancreatic cancer, breast cancer, prostate cancer, lung cancer (including small-cell lung carcinoma and non-small-cell carcinoma, leukemia, lymphoma, sarcoma, ovarian cancer, Kaposi's sarcoma, Hodgkin's Disease, Non-Hodgkin's Lymphoma, multiple myeloma, neuroblastoma, rhabdomyosarcoma, primary thrombocytosis, primary macroglobulinemia, small-cell lung tumors, primary brain tumors, stomach cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, cervical cancer, endometrial cancer, glioblastoma, and adrenal cortical cancer. In some aspects, the cancer is a primary cancer. In some aspects, the cancer is metastatic.

The efficacy of treatment of a cancer may be assessed by any of various parameters well known in the art. This includes, but is not limited to, determinations of a reduction in tumor size, determinations of the inhibition of the growth, spread, invasiveness, vascularization, angiogenesis, and/or metastasis of a tumor, determinations of the inhibition of the growth, spread, invasiveness and/or vascularization of any metastatic lesions, determinations of tumor infiltrations by immune system cells, and/or determinations of an increased delayed type hypersensitivity reaction to tumor antigen. The efficacy of treatment may also be assessed by the determination of a delay in relapse or a delay in tumor progression in the subject or by a determination of survival rate of the subject, for example, an increased survival rate at one or five years post treatment. As used herein, a relapse is the return of a tumor or neoplasm after its apparent cessation, for example, such as the return of leukemia.

Cancers to be treated may include cancers that express the ATB^{0,+} transporter. Such expression may be at a level that is increased or enhanced when compared to the level expressed on normal, non-cancerous cells. Cancers to be treated may also include cancers that do not express the ATB^{0,+} transporter. The methods of the present disclosure may include a step of determining the level or amount of the ATB^{0,+} transporters expressed on cells in a sample and the comparison to expression levels of the transporter on normal or control cells. The methods of the present disclosure may include deciding to clinically treat a subject with the administration of alpha-methyl-tryptophan and further therapeutic agents if the cells in the sample express an increased level of the ATB^{0,+} transporter.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### α-methyl-L-tryptophan (α-MLT) is a Potent Inhibitor of Indoleamine Dioxygenase

Indoleamine 2,3-dioxygenase (IDO) is an important component of tumor-associated immune tolerance (Mellor and Munn, 2008, Nat Rev Immunol; 8:74-80). Tumors evade the surveillance of the immune system by inducing IDO in tumor cells themselves, stromal cells, or immune cells present in the tumor-draining lymph nodes. It is believed that tumor-associated up-regulation of IDO leads to enhanced breakdown of the essential amino acid tryptophan from the surroundings, with the resultant tryptophan depletion leading to prevention of T cell activation. Thus, induction of IDO is a critical determinant of immune tolerance towards tumors. Inhibition of IDO reverses this process, causing activation of the immune system and killing of tumor cells. 1-methyltryptophan (1-MT) is widely used as a pharmacologic inhibitor of IDO and is currently in preclinical development phase for treatment of cancer (see, for example, Hou et al., 2007, Cancer Res; 67: 792-801).

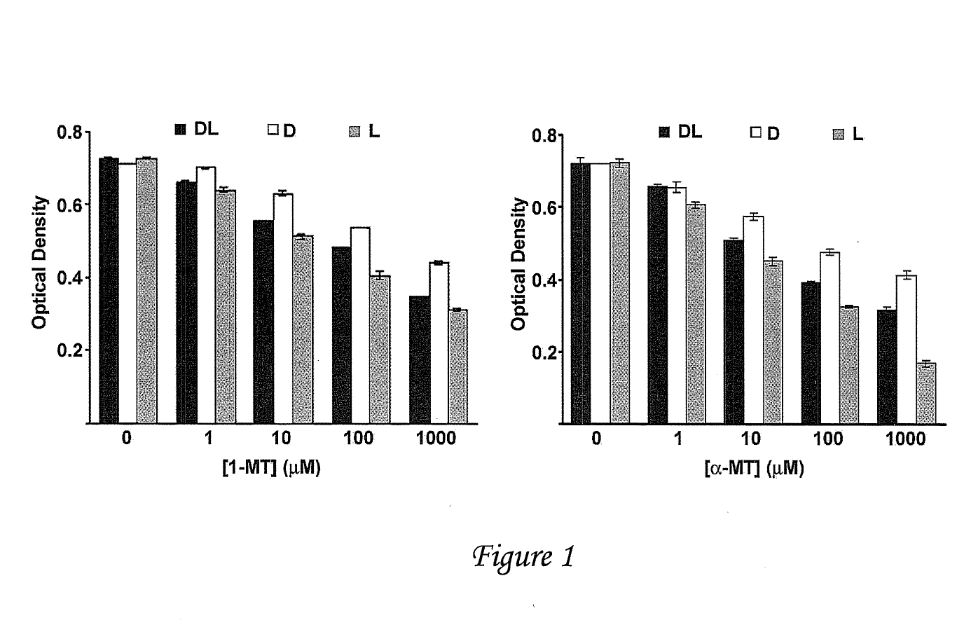

The present example demonstrates, for the first time, that α-methyltryptophan is also an inhibitor of IDO and that its potency as an IDO inhibitor is significantly greater than that of 1-methyltryptophan. FIG. 1 presents the comparative potencies of D-, L-, and DL-isomers of 1-methyltryptophan (1-MT) and alpha-methyltryptophan (alpha-MT) for inhibition of indoleamine dioxygenase (IDO). Furthermore, the L-isomer of α-methyltryptophan (α-MT) is significantly more potent than the D-isomer as an inhibitor of IDO. In these studies, mouse intestinal mucosal lysates were used as the source of IDO in a cell-free assay system. Thus, α-MLT will produce anti-cancer effects in vivo by two different, but synergistic, mechanisms. It will block the transport function of ATB^{0,+} in tumor cells, thus preventing entry of essential amino acids and glutamine, and consequently causing growth arrest and apoptosis. It will also inhibit IDO in tumor-associated immune cells, thus disrupting tolerance toward tumors and consequently triggering anti-tumor immune responses.

### Example 2

### Inhibition of T Cell Proliferation

This example complements the cell-free in vitro measurements of IDO activity shown in Example 1 with a functional assay involving T cell proliferation. In this assay, CD8⁺ T cells from a transgenic mouse (01.1) which carries the T-cell receptor specific for the ovalbumin peptide antigen Ser-Ile-Ile-Asn-Phe-Glu-Lys-Leu (SIINFEKL) were used. CD11c⁺ dendritic cells (DCs) were also prepared from a C57B16 mouse that has been given CpG 1826 intravenously to induce IDO in DCs. A mixed lymphocyte reaction was then performed in which the ability of IDO-positive DCs to suppress the proliferation of CD8⁺ T cells was monitored using [³H]thymidine incorporation as the read-out. Thymidine incorporation in these T cells in the absence of DCs was taken as the control. When the T cells were cultured in the presence of IDO-positive DCs (10⁵ cells), thymidine incorporation in T cells was suppressed by 63±3%. This effect was completely reversed in the presence of 0.2 μM α-MLT (racemic mixture). It is important to note that the concentration of α-MLT needed to inhibit IDO in DCs in this cell-based ex-vivo assay system (0.2 μM) is almost two orders of magnitude less than the IC₅₀ observed for the inhibition of IDO in the cell-free assay system (˜20 μM). Based on these findings, it is expected that α-MLT will function as an anti-cancer agent in vivo by acting through two different mechanisms. One, by blocking the tumor-specific nutrient transporter ATB^{0,+} thereby starving the tumor cells of essential amino acids, and two, by also inhibiting IDO thereby enhancing the activity of the immune system against the tumor.

### Example 3

### α-methyltryptophan as a Blocker of ATB^{0,+}

Tryptophan is a substrate for ATB^{0,+}. While screening a variety of tryptophan derivatives for interaction with ATB^{0,+}, it was found that α-methyl-DL-tryptophan (α-MT) inhibited ATB^{0,+}-mediated glycine uptake but did not induce inward currents in ATB^{0,+}-expressing Xenopus oocytes (Karunakaran et al., 2008, Biochem J; 414: 343-355). Inhibition of glycine uptake could occur because α-MT is a transportable substrate for ATB^{0,+} thus competing with glycine for the uptake process, or because it blocks the transporter without itself being transported. The Xenopus oocyte expression system can differentiate between these two modes of inhibition because transportable substrates would induce inward currents whereas blockers would not. The inability of α-MT to induce currents indicates that it functions as a blocker of ATB^{0,+}.

Next, the potency of α-MT to block the transporter under conditions that simulate those in vivo was determined (with all naturally occurring amino acids present at physiologic concentrations). ATB^{0,+} accepts all proteinogenic amino acids except glutamate and aspartate. Therefore, using the Xenopus oocyte expression system, the transport activity of human ATB^{0,+} was monitored in the presence of an amino acid mixture consisting of all proteinogenic amino acids, each at its respective physiologic concentration found in plasma. Inward currents were detected under these conditions in oocytes expressing human ATB^{0,+}. α-MT blocked the ATB^{0,+}-mediated inward currents in the presence of these amino acids. The inhibition was dose-dependent; the concentration of the blocker necessary to elicit 50% maximal inhibition was 255±24 μM (Karunakaran et al., 2008, Biochem J; 414: 343-355).

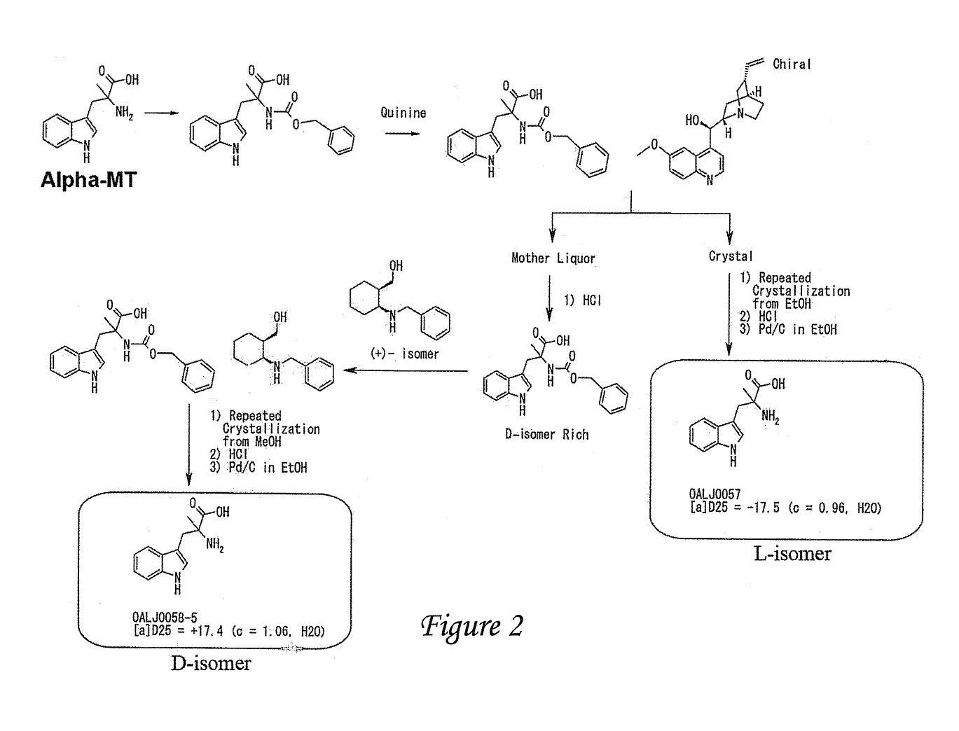

α-Methyl-L-tryptophan (α-MLT) is more potent than α-methyl-D-tryptophan as a blocker of ATB^{0,+}. Although α-methyl-DL-tryptophan is a blocker of ATB^{0,+}, its potency is weak. This example demonstrates that the L-isomer of α-methyltryptophan is a more potent blocker than the D-isomer. The only commercially available α-methyltryptophan is the DL-form. Thus, an optical resolution method was developed to separate the L-isomer from the DL-form (FIG. 2). With this method, the L- and D-isomers of this compound were purified. The potencies of the two isomers as a blocker of ATB^{0,+} was examined in Xenopus oocytes using the amino acid mixture simulating the plasma. The concentration of the L-isomer to elicit 50% maximal inhibition was 18±5 μM. This value is 14 times less than the corresponding value for the DL-form. With the D-isomer, the inhibition never increased beyond 40% even at 1 mM.

### Example 4

### In Vivo Anti-Tumor Effect

Following procedures similar to those described in Hou et al. (Hou et al., (2007) “Inhibition of indoleamine 2,3-dioxygenase in dendritic cells by stereoisomers of 1-methyl-tryptophan correlates with antitumor responses,” Cancer Res. 67: 792-801) and WO 2010/02685, the in vivo anti-tumor effect of alpha-methyltryptophan in mouse models of transplantable melanoma, transplantable colon cancer, and transplantable breast cancer will be determined. Cancers tested will include cancers that do not expresses the ATB^{0,+} transporter and cancers that express the ATB^{0,+} transporter. Cancers tested may represent an autochthonous cancer and/or metastatic cancers. Alpha-methyltryptophan will be administered in the drinking water and/or with implantable subcutaneous pellets. Preparations of the racemic D/L mixture, the D-isomer, and/or the L-isomer of alpha-methyltryptophan will be tested.

### Example 5

### Inhibition of Tumor Growth In Vivo

Mouse xenograft studies were performed with two ER-positive breast cancer cell lines (ZR75.1 and MCF7) and one ER-negative breast cancer cell line (MB231). Alpha-methyltryptophan in drinking water (2 mg/ml) reduced growth of ZR75.1 cells but did not effect the growth of MCF7 cells (in the presence of estrogen administration) and MB231 cells. Plasma concentration of alpha-methyltryptophan in mice after two weeks of administration in drinking water was 8.5±0.5 Alpha-methyltryptophan was not detectable in control mice.

Further, alpha-methyltryptophan was administered in a mouse model of colon cancer. Following subcutaneous injection of mouse colon cancer cells into mice, mice were treated with alpha-methyltryptophan, either in drinking water or by intraperintoneal injection. In both cases, the growth of the tumor was reduced significantly (approximately 50%).

### Example 6

### Synergistic Effect

Following procedures similar to those described in U.S. Patent Applications 2004/0234623 and 2005/0186289, and by Hou et al., 2007, Cancer Res; 67: 792-801) the cooperativity effect of alpha-methyltryptophan with chemotherapy and radiation will be determined. Preparations of the racemic D/L mixture, the D-isomer, and/or the L-isomer of alpha-methyltryptophan will be tested.

### Example 7

### Allogeneic Mixed Lymphocyte Cultures

Following procedures similar to those described in Hou et al., 2007, Cancer Res; 67: 792-801, the effect of alpha-methyltryptophan on human and murine allogeneic mixed lymphocyte cultures will be determined. Preparations of the racemic D/L mixture, the D-isomer, and/or the L-isomer of alpha-methyltryptophan will be tested.

### Example 8

### Immunomodulation

Following procedures similar to those described in WO 2007/050405, WO 2007/081878, and WO 2008/100691 the immunomodulatory effect of alpha-methyltryptophan on TLR9 ligand IDO induction and T cell suppression, generation of regulatory T cells, and the interaction of IDO, PD-1/PD-L pathways and CTLA4 pathways in the activation of regulatory T cells will be determined. Preparations of the racemic D/L mixture, the D-isomer, and/or the L-isomer of alpha-methyltryptophan will be tested.

### Example 9

### Human Clinical Trials

Human trials assessing the suitability of alpha-methyltryptophan as an IDO inhibitor to block host-mediated immunosuppression and enhance antitumor immunity in the setting of combined chemo-immunotherapy regimens will be undertaken. Preparations of the racemic D/L mixture, the D-isomer, and/or the L-isomer of alpha-methyltryptophan will be utilized.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims. All headings are for the convenience of the reader and should not be used to limit the meaning of the text that follows the heading, unless so specified.

1. A method of inhibiting indoleamine-2,3-dioxygenase (IDO), the method comprising contacting the IDO with a composition comprising alpha-methyl-tryptophan.

2. A method of inhibiting indoleamine-2,3-dioxygenase (IDO) in a subject, the method comprising administering an effective amount of a composition comprising alpha-methyl-tryptophan.

3. (canceled)

4. The method of claim 2 wherein the inhibition of IDO results in increased T cell activation by an antigen-bearing cell, an enhanced immune response, reduced immune suppression mediated by regulatory T cells (Tregs), and/or an enhanced T cell mediated immune response in the subject.

5. (canceled)

6. A method to enhance an immune response to an antigen in a subject, the method comprising administering to the subject an effective amount of such an antigen in combination with alpha-methyl-tryptophan.

7-8. (canceled)

9. A method of treating a subject with an infection, the method comprising administering to the subject an effective amount of a composition comprising alpha-methyl-tryptophan.

10-12. (canceled)

13. The method of claim 2, wherein the subject is an individual with cancer, an individual receiving a bone marrow transplant or peripheral blood stem cell transplant, or an individual already undergoing a standard antiretroviral HIV therapy.

14. The method of claim 13, wherein the cancer is selected from the group consisting of melanoma, colon cancer, pancreatic cancer, breast cancer, prostate cancer, lung cancer, leukemia, brain tumors, lymphoma, sarcoma, ovarian cancer, and Kaposi's sarcoma.

15. (canceled)

16. The method of claim 13, wherein the cancer expresses the ATB^{0,+} transporter.

17. The method of claim 2, wherein the alpha-methyl-tryptophan consists of the isolated D isomer, consists of the isolated L isomer, or comprises racemic mixture.

18. The method of claim 2, wherein the alpha-methyl-tryptophan comprises the D isomer of alpha-methyl-tryptophan and does not comprise the L isomer of alpha-methyl tryptophan or comprises the L isomer of alpha-methyl-tryptophan and does not comprise the D isomer of alpha-methyl tryptophan.

19. (canceled)

20. The method of claim 2, wherein the alpha-methyl-tryptophan is formulated for controlled or sustained release, for enteral administration, for systemic administration, or for topical administration.

21. The method of claim 20, wherein the formulation for controlled or sustained release is suitable for subcutaneous implantation or is a patch.

22-26. (canceled)

27. The method of claim 2, wherein the subject is an individual with HIV.

28. The method of claim 2 further comprising the administration of an additional therapeutic agent.

29. The method of claim 28 wherein the additional therapeutic agent is selected from the group consisting of an antiviral agent, an antibiotic, an antimicrobial agent, a cytokine, a vaccine, an antineoplastic chemotherapy agent, radiation therapy, and combinations thereof.

30. The method of claim 28, wherein the administration of alpha-methyl-tryptophan and the at least one additional therapeutic agent demonstrate therapeutic synergy

31-40. (canceled)

41. The method of claim 28, wherein at least one additional therapeutic agent is an inhibitor of indoleamine-2,3-dioxygenase (IDO) other than alpha-methyl-tryptophan or an inhibitor of the ATB^{0,+} amino acid transporter other than alpha-methyl-tryptophan.

42. (canceled)

43. The method of claim 28, wherein at least one additional therapeutic agent is 1-methyl-tryptophan (1-MT).

44. The method of claim 43, wherein the 1-MT consists of the isolated D isomer of 1-MT, consists of the isolated L isomer of 1-MT, or comprises a racemic mixture of 1-MT.

45-55. (canceled)

56. The composition of claim 59, wherein the alpha-methyl-tryptophan consists of the isolated D isomer, consists of the isolated L isomer, or comprises a racemic mixture.

57-58. (canceled)

59. A composition comprising alpha-methyl tryptophan and at least one additional therapeutic agent.

60. The composition of claim 59, wherein the at least one additional therapeutic Agent comprises an inhibitor of indoleamine-2,3-dioxygenase other than alpha-methyl-tryptophan.

61. The composition of claim 59, wherein the at least one additional therapeutic agent comprises 1-methyl-tryptophan (1-MT).

62. The composition of claim 61, wherein the 1-MT consists of the isolated D isomer of 1-MT, consists of the isolated L isomer of 1-MT, or comprises a racemic mixture of 1-MT.

63-70. (canceled)
